(19) United States
(12) Patent Application Publication
(10) Pub. No.: US 2006/0093691 A1
(43) Pub. Date: May 4, 2006

(54) Composition for oral or corporeal hygiene or for health comprising seeds or ground material or protein extracts based on seeds of a plant of the genus moringa

(76) Inventors: Phillipe Jean Louis Thurot, Saintry (FR); Morin Chhun, Evry (FR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/516,057

(22) PCT Filed: May 28, 2002
(86) PCT No.: PCT/FR02/01780

§ 371 Date: Sep. 22, 2005

(30) Foreign Application Priority Data
May 29, 2001 (FR) 01/06957

(51) Int. Cl. A61K 36/60

(52) U.S. Cl. 424/776

(57) ABSTRACT

The invention concerns a deodorant composition of a composition for oral or corporeal hygiene, or for health, characterized in that it comprises seeds of a plant of the genus Moringa as such or ground or in the form of a protein extract.

### SCOPE OF INVENTION

This invention pertains to the areas of cleanliness, health, cosmetics, dermatology, body hygiene, buccal and dental hygiene. It relates to compositions used in these areas and characterized by the fact that they contain seeds or crushed seeds or a protein extract or extracts from seeds from a plant of the Moringa type.

### STATE OF TECHNIQUE

It is known that the MORINGA type contains more than ten plant species throughout the world.

It is known that the MORINGA type corresponds to a fast-growth, falling-leaf bush that proliferates in the tropics, in Asia, Africa, and South America.

The fruit of the MORINGA-type plants form pods about thirty to fifty centimeters long, which are usually eaten green, like vegetables, and which produce seeds.

The various parts of the bush are used in conventional medicine.

The MORINGA seeds contain in particular an oil known as Ben oil or Behen oil which was for a long time used for the formulation of ointments used for cosmetic and religious purposes.

The seeds are also used for the clarification of water and it is also known that they contain flocculant compounds and have anti-bacterial powers.

PCT application no. WO 98/37863, published on Sep. 3, 1998, mentions the use of Moringa oil for the purpose of improving the anti-fungus and anti-infection effects of oil from plants of the Calophylum type. This document describes no composition containing seeds, crushed seeds or a protein extract from a plant of the Moringa type nor, all the more, their applications.

Also known, through the publication of French patent application no. 2 776 519, is the use of certain protein fractions derived from Moringa seeds and delipidated and purified by cation-exchange chromatography for depolluting skin, and more specifically for eliminating, by capture, particles such as plant carbon. In view of this document, people working in the field are dissuaded from implementing seeds or crushed seeds of the Moringa type, without prior extraction or protein fractioning, for a skin-depollution application.

### DETAILED DESCRIPTION OF INVENTION

According to the invention, it is henceforth shown that MORINGA seeds, whole or crushed, decorticated or un-decorticated, or which were crushed or from which were made delipidated or non-delipidated protein extracts, have new, original and beneficial properties in the areas of deodorization and the elimination of offensive odors, cleanliness, hygiene for personal parts, buccal hygiene and dental care related to mouth health, body care and hygiene, and health, allowing them to be used directly in cosmetics, pharmacology, as a health food, as hygienic and cleanliness products, and as beauty and skin care products.

More specifically, it is shown according to the invention that seeds of a Moringa-type plant, or else crushed seeds or a protein extract from these seeds have the property of destroying offensive body and mouth odors; preventing and limiting the appearance of offensive body and mouth odors.

It is also shown according to the invention that seeds of a Moringa-type plant, or else crushed seeds or a protein extract from these seeds have the property of favoring salivation; preventing the formation of tooth film and tartar and helping to eliminate them; preventing the appearance of caries; preventing the appearance of aphthas; favoring the healing of aphthas, necroses and mouth sores and quickly reducing the pain related to these aphthas, aphtoses and mouth sores.

The composition used for buccal or body hygiene, and/or cleanliness, and/or health care, according to the invention, can also be characterized by the fact that the seeds are decorticated or not decorticated.

Thus provided, according to the invention, are compositions or formulations used for buccal or body hygiene, hygiene for personal parts, cleanliness, dental care, skin care and personal hygiene, general personal care, and health care, characterized by the fact that they contain seeds or crushed seeds or a protein extract from a Moringa-type plant, especially from a plant of the Moringa oleifera species.

Everything discussed so far means that the Moringa seeds, the crushed seeds and their protein extract find many applications in the area of body deodorization, room deodorization and in particular deodorization of feet, arm pits and other parts of the human body yielding offensive odors.

It was also shown that it was not imperative to extract the oil contained in the Moringa seeds in order to obtain the maximum beneficial effects related to the deodorizing and body-odor destroying properties. A protein extract whose oil has been remove has likewise a body-odor destroying and/or deodorizing property, but that this new property was less durable and effective over time.

The Moringa seeds, the crushed seeds and their protein extract are also useful in applications for buccal and dental hygiene and care; for the prevention of tartar, tooth film and caries and the fight against them; for dental and buccal care and hygiene; for, in particular, the elimination of bad breath, with quick destruction of offensive odors or bad breath and/or maintaining of a fresh breath; for helping salivation; for preventing the appearance of aphthas, aphtoses and helping to heal them and reducing the pain related to aphthas, aphtoses and other sores.

As examples illustrating this discovery (non-exhaustive), described below are various applications and processes for obtaining preparations or formulations or products which can be used and marketed within the framework of this invention.

Consequently, another purpose of this invention is to provide a cosmetic and/or pharmaceutical composition and/or used for body care and/or used for mouth and dental care and/or feminine or masculine hygiene or related to children and older people and/or incontinence; and or the manufacture of odor-proof fabric and clothing and products; and/or as products for fighting buccal aphthas, aphtoses, sores and ulcers; in particular used as a deodorant for the human body (lotions, creams, foams, deodorant or odor-destroying products); in particular as products for reducing or eliminating bad breath and/or providing a fresh breath.

Another purpose of this invention is to provide compositions used for reducing or eliminating tartar and tooth film; as products producing extensive salivation and eliminating the negative effects of a dry mouth, including use with persons suffering from hyposyalia and xerostomia; products having a beneficial effect on the prevention of caries; products speeding up the healing of sores, necroses, mouth ulcers and aphtoses; products reducing pain related to aphthas, necroses, mouth ulcers and sores; products for preventing and fighting aphtoses.

According to the invention, the compositions as defined in a general manner above are characterized by the fact that they contain, as the active constituent, Moringa seeds or crushed seeds, whose oil has been removed or not removed, or a protein extract, whose oil has been removed or not removed, extracted from the seeds of a MORINGA-type plant belonging to the family of Moringaceae.

Preparation of Seeds, Crushed Seeds and Proteinaceous Seed Extracts.

Decortication and Crushing of Moringa Seeds

In conformity with a preferred way of producing the invention, the MORINGA seeds are crushed; these can be decorticated or undecorticated, i.e. have their protective covering removed or unremoved; these crushed seeds can have their oil removed or not removed.

As an illustrative and non-exhaustive example, a process for decorticating and crushing the seeds will be described below.

The method for decorticating the seeds consists of using a mechanical process designed to first screen the seeds by size if there are different sizes. The method then consists of crushing each category of seeds screened, between two rigid plates, leaving a space between the plates that is two, three, or four millimeters less than the size of the mesh used for prior screening of the seeds. The result of the process is to break up the integumentary husks of the seeds, without destroying the almond that each seed contains. As the third step, the integument which is broken up, and lighter than the almond, is separated from the almond by a system of screening and vibration-type sifting. The almond can then be crushed with a mechanical crushing machine.

Removing Oil or Lipid from Moringa Seeds or Crushed Seeds

Furthermore, the crushed seeds can have their oil removed or not removed.

As illustrative and non-exhaustive examples, various processes for extracting the oil from the seeds will be discussed below.

The oil can be eliminated by extraction with organic solvents or steam distillation.

The oil can also be extracted by pressure on a type of press known as the Komet press.

Obtaining a Powder from Crushed Seeds

In addition, the crushed seeds can be converted to a powder.

As illustrative and non-exhaustive examples, various processes for producing the powder, well known to people in the field, will be discussed below.

The seed powder can be obtained, in particular, through cryogenic techniques. It can also be obtained through cryo-crushing or vacuum lyophilization.

Preparation of a Protein Extract from Moringa Seeds

As illustrative and non-exhaustive examples, several process, well known to people in the field, for producing protein extracts which can be used within the framework of this invention will be described below.

According to the first process, the almonds obtained can first have their lipid removed by one or several successive reflow extractions in a solvent such as hexane, and after filtration, have their flour dried in a stove.

Stove drying can be carried out at various temperatures reaching, in particular, 40° C.

According to a second process, the protein extracts can also be obtained after mixing the flour with distilled or spring water in order to obtain an aqueous extract, then centrifuged, filtered and dehydrated.

According to a third process, a precipitate can also be obtained from the aqueous extract. It can first be cleaned in order to remove any impurities, then dissolved again, then centrifuged so that a protein precipitate is obtained.

According to a fourth process, the protein extracts can also be obtained after first producing an aqueous extract then decanting.

As illustrative and non-exhaustive examples, the seeds, crushed seeds and seed and powder extracts can be stored either by stove drying, cryogeny or vacuum lyophilization; techniques well known to people in the field.

Deodorant or Deodorizing Compositions

The invention also concerns the use of seeds, crushed seeds or proteinaceous seed extract or a mixture of seeds, crushed seeds or proteinaceous seed extract from a MORINGA-type plant, belonging to the Moringaceae family, as the active constituent, alone or as a mixture, alone or associated with at least one other active constituent, for the preparation of a composition or a formulation able to be used as a deodorant or deodorizer and/or hygiene products and/or related to cleanliness for personal parts which eliminates offensive odors or limits their appearance.

According to the invention, it is shown that the seeds, crushed seeds or proteinaceous seed extracts, whose oil has been removed or not been removed, decorticated or not decorticated, present, in particular, deodorant and deodorizing activities, and activities eliminating offensive body odors with an additional soothing, softening, and hydrating effect on the skin, and reducing the sensation of fatigue; that they also present deodorant and deodorizing activities and activities destroying unpleasant odors with clothing, fabric, shoes and any materials in contact with skin and sweat.

It is shown in the examples that a deodorant or deodorizing composition based on Moringa seeds was particularly effective for maintaining body hygiene throughout the day.

First of all, a composition based on crushed Moringa seeds shows itself to be very effective with respect to offensive odors, while the effectiveness of a conventional perfumed deodorant is mediocre.

Moreover, the results presented in the examples show that a deodorant composition based on crushed Moringa seeds is at least as effective as a perfumed deodorant containing a bactericidal additive from synthetics chemistry such as trichlosan.

It is known that Moringa seeds, the crushed seeds or else a protein extract from these seeds consist of a natural substance, not causing allergy, not having the potential drawbacks for health found in synthetics. In addition, this natural substance, easily and quickly prepared, is significantly less costly than the synthetic products currently used, in particular anti-bacteria or anti-fungus additives.

On the basis of preliminary experimental results, the applicant thinks, without wanting to be bound to any kind of theory, that the anti-odor properties of the Moringa seeds, the crushed seeds or the protein extracts from these seeds are not due to any kind of anti-bacteria or anti-fungus effect, but rather to an odor-trapping effect. If that proved to be the case, another technical advantage would result from the deodorant or deodorizing compositions, according to the invention, namely maintaining of the bacterial and fungal flora balance present at the surface of the mucous membranes, and more specifically of the epidermis, which cannot be kept intact when conventional deodorants containing anti-bacteria and anti-fungus compounds are used.

According to the first specific method of production, a deodorant or deodorizing composition, according to the invention, contains the Moringa seeds, or the crushed seeds or a protein extract from Moringa seeds associated with at least one anti-bacteria compound and/or with at least one anti-fungus compound, for example a compound conventionally used in this type of composition, in order to combine the Moringa seeds' capacity for trapping odors with the capacity of reducing the undesirable development of bacteria and fungus, which is in particular a source of unpleasant odors.

According to a second specific method of production, a deodorant or deodorizing composition, according to the invention, will contain no anti-bacteria compound, and, should this be the case, no anti-fungus compound, associated with Moringa seeds, the crushed seeds or the protein extract from these seeds. On the basis of preliminary experimental results, the applicant thinks, without wanting to be bound to any kind of theory, that the effect of the anti-bacteria compounds, and, should this be the case, the effect of the anti-fungus compounds, are not added to the effects obtained with the Moringa seeds, the crushed seeds or their protein extract. In this particular method of producing a deodorant or deodorizing composition, according to the invention, the said composition contains no anti-bacteria or anti-fungus compound exogenous to the Moringa seeds, the crushed seeds or their protein extract.

According to still another specific method of production, a deodorant or deodorizing composition, according to the invention, especially when it is presented in the form of a composition for fighting perspiration, also contains at least one anti-perspiration compound or agent, such as aluminum chlorohydrate or triethyl acetate.

It is also shown, according to the invention, that the seeds, the crushed seeds and the proteinaceous Moringa extracts, with their oil removed or not removed, have cosmetic properties that are softening, hydrating, soothing and fatigue-relieving with respect to the skin, giving the skin and skin outgrowths a soft and pleasant touch; this, in conjunction with the deodorant and deodorizing properties, allows them to be used with a double advantage in cosmetics and/or therapy, used locally for the skin and/or skin outgrowths.

Furthermore, it is shown that when the seeds or the crushed seeds or the seed extracts do not have their oil removed, their softening, hydrating, soothing and fatigue-relieving properties are improved on the skin and the skin outgrowths, which increases their cosmetic and therapeutic interest as a deodorant and a deodorizer.

It has been shown, according to the invention, that the seeds, the crushed seeds or a seed extract have high deodorant and/or deodorizing power.

The new and beneficial properties result from the observation, conducted with a large number of persons, that crushed seeds and/or a protein extract from MORINGA seeds provide quick and durable elimination of offensive body odors; that they also keep these unpleasant odors, resulting from perspiration, from returning.

The observation has been made that, by mixing the crushed seeds or a MORINGA seed extract with liquids such as water or with creams or milk fluids, as soon as any part of the body having an offensive odor is dipped into the mixture produced, such as feet; or when the mixture is used to wash, for example, the arm pits or the crotch, the unpleasant body odors disappear immediately and permanently and lastingly.

It has, once again, been observed that the crushed seeds or seed extract, with their oil removed or not removed, mixed with water, produced a milky emulsion cream white in color which did not foam; this allowed this product to be used as the equivalent of a natural milk fluid; without also having to add to the mixture alcohols, solvents and any other anti-foaming products; and that it also did not have the traditional disadvantages of soap (in particular, the causticity of soap . . . ); that consequently, the crushed seeds or proteinaceous seed extract, with its oil not removed or removed, could be perfectly incorporated into the composition of soap and any other cosmetic preparation to give it an additional deodorant and deodorizing property.

It has also been shown, according to the invention, that this suspension, associating either crushed seeds, or a seed extract associated with its oil, and consequently without its oil removed, mixed with water, also had hydrating, protective and soothing powers for skin, reducing the feeling of fatigue; that this suspension kept all its powers as a deodorant and destroyer of unpleasant odors; that it was therefore also possible to maintain, in particular, all the cosmetic properties of the Moringaceaes seeds (hydrating power, protective, soothing and fatigue-relieving powers), without having to necessarily extract the oil from these seeds; that it was thus possible to associate the crushed seeds or the Moringaceaes extracts with any cosmetic et pharmaceutical preparation, giving these preparations new and natural properties: soothing, softening, hydrating, fatigue-relieving, deodorant and skin-protection properties.

It has, in particular, been shown that by dipping the smelly feet of different persons into a container filled with this “milky” emulsion, associating crushed seeds or a protein extract from MORINGA seeds with spring water, then having the feet removed from this bath; and without rinsing them; the surface of the skin of the feet became particularly soft, silky and pleasant to touch; with an effect of softness comparable to that of a “baby's skin”; that the skin of the feet no longer smelled bad; that, deposited on the surface of the skin, was a thin, protective film, very soft and silky, originating from the crushed seeds or the proteinaceous seed extract; that this deposit enabled the feet to maintain for a long time (more than a day, even two or three days) the deodorant and offensive-odor-destroying properties of the product on the parts of the body having bathed in or been in contact with the solution; that this composition brought an improvement in terms of touch and softness of the skin, being pleasant and good for the skin; with, in particular, for persons, the feeling of having a clean and healthy skin; with, also for them, a considerable feeling of well-being and the observation on the skin of the softening, soothing, hydrating, fatigue-relieving, deodorant and deodorizing effect of the crushed seeds and the protein extracts from the MORINGA seeds with their oil removed or not removed.

It has also been shown that the crushed seeds or a MORINGA seed extract having this deodorant and odor-destroying power as such, presented no allergy, drying and caustic reaction on the skin; as different from other products such as alun (aluminum silicate) whose deodorant properties are known, but which, in particular, can dry out the skin and cause allergies.

A deodorant and/or deodorizing composition, according to the invention, can come in the form of a composition for fighting the consequences of perspiration and for destroying or limiting the appearance of unpleasant body odors.

A composition, according to the invention, can come in the form of crushed seeds, whose oil is removed or not removed, for making liquid or solid soap, skin-care bars, creams, gels, lotions and foams which are deodorant and/or deodorizing, soothing, softening, hydrating and fatigue-relieving, well known by the people working in the field.

Preferably, a deodorant or deodorizing composition, according to the invention, also contains a perfume, such as Madison perfume, for example.

A deodorant or deodorizing composition, according to the invention, can also contain an alcohol, such as ethanol, or compounds such as cyclomethicone pentamer and propylene glycol, in particular when the composition comes in a form suited for use as a spray.

According to another aspect, a deodorant composition, according to the invention, is characterized by the fact that it is a topic composition in the form of a liquid cleaning solution used as such or in the form of a cloth, a wad of cotton or a cellulose cleaning napkin or a fabric wetted with the deodorant and/or deodorizing solution; a milk fluid, a cream, a soap bar, a liquid or solid soap, a skin-care bar, a lotion or a foam.

The liquid forms can also and advantageously be dispensed by means of a spray can, a conventional applicator, and a roll-on applicator.

According to yet another aspect, a deodorant composition, according to the invention, is characterized by the fact that it is a powder, placed, if need be, in a container fitted with a dispersion mechanism or used as is; or used as a mixture for baths, for salts, talcs; or used in the composition of special products for preparing feet for physical activity or for sports; or used in the composition of dry shampoos.

A deodorant or deodorizing composition, according to the invention, can come in the form of a stick, a gel or a bi-gel.

According to another advantageous characteristic of a composition according to the invention, such a composition comes in the form of a milk fluid, a cream or a soap bar. For the preparation of such deodorant and/or deodorizing compositions according to the invention, people working in the field can advantageously refer to American patents U.S. Pat. No. 3,932,609, U.S. Pat. No. 3,949,066, U.S. Pat. No. 3,974,270, U.S. Pat. No. 4,049,792 and U.S. Pat. No. 4,045,548.

For fabricating a composition according to the invention in the form of a soap bar or other form of soap, people working in the field can advantageously refer to American patents U.S. Pat. No. 5,952,276, U.S. Pat. No. 5,952,275 and U.S. Pat. No. 5,874,392.

A composition according to the invention can come in the form of a powder which can be incorporated into various preparations as a mixture, for baths, salts, talcs and dry shampoos.

The powder can be placed in a container fitted with a mechanism for dispersing the powder, or “spray”, also well known to people in the field.

A composition according to the invention can come in the form of crushed seeds or a seed extract, whose oil has been removed or not removed, mixed into deodorant products, and in particular, deodorant sticks and gels well known to people in the field.

A composition according to the invention can come in the form of a deodorant stick in the form of a bi-gel which would contain the fatty substances and enough water to hydrate the area of application. This bi-gel can then be the combination of an aqueous gel based on cellulose derivatives or polymethacrylics and a gel based on vegetable oils and ethylcellulose.

A composition according to the invention can come in the form of crushed seeds or a seed extract or a powder combined with an effervescent formula such as an effervescent powder or an effervescent tablet favoring dispersion of the product for bathing the feet and/or for washing.

The product would come in the form of an effervescent structure obtained by compression.

The seed powder will, in particular, be added to the effervescent mixture by means of various methods of granulation well known to the people in the field, such as that obtained by a dry process, a wet process or by granulation on a fluidized layer of air.

A deodorant or deodorizing composition according to the invention can be a semi-fluid, emulsive preparation containing a suspension of seed powder, and which can, in particular, be used as an odor-fighting and fatigue-relieving product. This emulsion would be, preferably but not in a limited way, based on different fatty substances, compatible with the product and with a high percentage of water, able to reach 90%, for, in particular, favoring humidification of the parts of the body in contact with the product and facilitating the activity of the preparation. This preparation can be used for the whole body, but also more particularly for the feet or the arm pits or the parts of the body generating bad odors.

A composition according to the invention can come in the form of crushed seeds or a powder mixed into the absorbent and/or deodorant raw materials used for making diaper pants, protectors for personal parts and sanitary napkins and/or related to incontinence.

A deodorant composition according to the invention can come in the form of a product entering into the composition of fabrics, clothing, and any anti-odor material, and can be incorporated into these products by, in particular, impregnation.

A composition according to the invention can come in the form of a protein extract in the form of an encapsulated composition allowing the encapsulation molecules to be incorporated into fabrics for making clothes, products and materials which will have less tendency to retain unpleasant body odors.

A composition according to the invention can come in the form of a liquid cleaning solution used as such or in the form of a cloth, a wad of cotton or a cellulose cleaning napkin or a fabric impregnated with the deodorant and/or deodorizing solution.

The crushed seeds or the MORINGA seed extracts according to the invention can thus be used for applications related to room deodorization or the elimination of offensive body odors with additional hydrating, soothing and fatigue-relieving effects; and/or for cleanliness and hygiene for personal parts; and/or for body and skin care; and/or for buccal and dental care and hygiene; and/or for health care.

They can also be used as products for fighting unpleasant foot, arm pit and body odors; for masculine and feminine hygiene; for the hygiene of sportsmen and people involved in activities causing perspiration; for babies' diapers and sanitary napkins and underpants/panties protectors or pants protectors or incontinence, mixed with absorbent and/or deodorant and deodorizing products forming them.

They can be used yet again for applications related to miscellaneous washing and cleaning products, without having the effects of conventional soaps.

They can, lastly, be incorporated into the fibers of fabrics for making smart fabrics, clothing and products which will limit the development of offensive upon contact with the skin.

Compositions for Buccal Hygiene

It is also shown according to the invention that seeds, crushed seeds or Moringa seed extracts also present a beneficial activity favoring and/or improving buccal salivation.

It is likewise shown according to the invention that the seeds, the crushed seeds and seed extracts also present a powerful activity as a deodorant and for destroying unpleasant odors and bad breath from the mouth.

It is also shown according to the invention that this salivation and the seeds, crushed seeds or seed extracts as such, help eliminate tartar and tooth film, making them more particularly suitable for preventing the appearance of caries.

It is also shown according to the invention that seeds or crushed seeds or MORINGA seed extracts also present an interesting activity for preventing the appearance of aphthas, aphtoses, ulcers and other mouth sores.

It is also shown according to the invention that de Moringa seeds or crushed seeds or extracts from these seeds have the property of helping to heal aphthas, ulcers and mouth sores; and are also advantageous for reducing the pain related to aphthas, ulcers and mouth sores.

Compositions for Buccal Hygiene Favoring Salivation and their Applications

It has also been shown that the seeds, the crushed seeds or a MORINGA seed extract help salivation, which can have considerable beneficial effects on health and buccal hygiene.

Observation of a considerable increase in buccal salivation was evaluated with various people, with the sugar test. This test consists of allowing to melt under the tongue a no. 4 lump of sugar. With most people in good health, the sugar normally melts under the tongue in less than three minutes. By consuming a seed, crushed seeds or a Moringa seed extract, this same lump of sugar melts in half the time, and salivation lasts well after the seeds, the crushed seeds or the extract has been thrown away or consumed, including people who have difficulty in salivating.

This improvement in salivation has beneficial consequences concerning health and buccal hygiene.

It is indeed known that a lack or insufficiency of saliva (hyposialia or xerostomia) can have harmful consequences for the state of one's health such as the appearance of gingivitis, recurrent buccal candidoses, dental complications or other complications involving the mouth, the tongue or the life of the patient; for example, due to the necessary modification of the food eaten compensating for the difficulties encountered in masticating, especially with aged people.

It is therefore clear that the increased salivation will have beneficial consequences in preventing all of these ailments.

Also shown according to the invention is the slightly bitter and sweet character, for taste, of the seeds, crushed seeds and MORINGA seed extracts, which makes the seed, the crushed seed or the seed extract directly assimilable and pleasant to eat, chew or suck on.

It is also an accepted fact that having a dry mouth also favors the development of bad breath.

As opposed to this, it is also an accepted fact that saliva modifies the PH of the mouth and helps eliminate food particles between the teeth; which also limits thoroughly the development of bad breath.

In accordance with the invention's preferred methods of production, the compositions produced favor and/or improve salivation. Such compositions according to the invention allow a fresh breath to be maintained.

A composition according to the invention can come in the form of a paste or chewing gum or tooth paste or a dentifrice enabling it to favor and/or improve salivation.

For preparing a composition according to the invention in the form of chewing gum, people working in the field can advantageously refer to American patents U.S. Pat. No. 3,984,574, U.S. Pat. No. 4,000,320, U.S. Pat. No. 4,151,270 and U.S. Pat. No. 4,415,593.

For preparing a composition according to the invention in the form of tooth paste, people working in the field can advantageously refer to American patents U.S. Pat. No. 3,932,606, U.S. Pat. No. 3,935,307, U.S. Pat. No. 3,941,877, U.S. Pat. No. 3,995,024, U.S. Pat. No. 4,181,712, U.S. Pat. No. 5,468,489 and U.S. Pat. No. 5,312,618.

A composition favoring and/or improving salivation in accordance with the invention can come in the form of a sucking candy.

A composition according to the invention can come in the form of a powder which can be incorporated into miscellaneous preparations and formulations.

The powder can be placed in a container fitted with a powder-dispersing mechanism or “spray”, likewise well known to people in the field.

Compositions for Buccal Hygiene Eliminating Bad Breath and their Applications

It has also been shown that Moringa seeds and/or the crushed seeds or a seed extract have a beneficial effect on the elimination of bad breath and allow a fresh breath to last longer, independently of the specific benefit for salivation with respect to bad breath.

It has also been observed that all persons who chewed or sucked on or masticated seeds or crushed seeds or a MORINGA seed extract, and who had bad breath, saw this bad breath disappear almost immediately; that if they chewed or masticated or sucked on the seeds or the crushed seeds or a seed extract in the evening, before going to bed, they no longer had such bad breath or else had no more bad breath at all in the morning when they got up.

A composition according to the invention can come in the form of a paste or chewing gum or tooth paste or a dentifrice for eliminating bad breath and allowing a fresh breath to last longer.

For preparing a composition according to the invention in the form of chewing gum, people working in the field can advantageously refer to American patents U.S. Pat. No. 3,984,574, U.S. Pat. No. 4,000,320, U.S. Pat. No. 4,151,270 and U.S. Pat. No. 4,415,593.

For preparing a composition according to the invention in the form of tooth paste, people working in the field can advantageously refer to American patents U.S. Pat. No. 3,932,606, U.S. Pat. No. 3,935,307, U.S. Pat. No. 3,941,877, U.S. Pat. No. 3,995,024, U.S. Pat. No. 4,181,712, U.S. Pat. No. 5,468,489 and U.S. Pat. No. 5,312,618.

A composition helping to eliminate bad breath and allowing a fresh breath to last longer according to the invention can come in the form of a sucking candy.

A composition according to the invention can come in the form of an emulsion for mouth washes, if they are necessary, or for improving the cleanliness of plates.

A composition according to the invention can be an emulsive, fluid or semi-fluid preparation containing a suspension of the seed powder, and which can, in particular, be used as a deodorant product for the mouth. This emulsion will be, preferably but not in a limited way, based on different oils fatty substances, compatible with the product and with a high percentage of water, able to reach 90%, for, in particular, favoring humidification of the parts of the mouth in contact with the product and facilitating the activity of the preparation. This preparation can be used as a gargle and possibly swallowed.

A composition according to the invention can come in a bioadhesive form with slow or fast disaggregation, as required, for releasing the quantity necessary for mouth deodorization. This composition could also be combined with an effervescent process.

A composition according to the invention can come in the form of crushed seeds or extracts or powders, dried or dehydrated or lyophilized, and can be placed in the mouth and chewed at the same time, before or after the person brushes his/her teeth with a dentifrice.

Compositions for Buccal Hygiene Helping to Eliminate Tooth Film and their Applications

It has also been shown that the seeds or the crushed seeds or a seed extract have the beneficial consequence of helping to eliminate tooth film and tartar and preventing caries; that, in addition, improving salivation also helps to eliminate bits of food that might be trapped between the teeth after a meal; that this also has the consequence of preventing effectively the occurrence of tooth film and tartar; and thus, indirectly, also preventing the appearance of caries, while at the same time also helping to eliminate bad breath.

It has consequently been shown that in a group of people who chewed, sucked on or masticated seeds or crushed Moringa seeds; that they had a cleaner and sounder dentition; that systematically the contact of their tongue with the surface of their teeth gave them the feeling of clean and smooth teeth, visibly cleaner, free of tooth film, and that the seeds or crushed seeds or a seed extract therefore necessarily had beneficial effects on the prevention of caries as it is an accepted fact that tooth film favors the formation of tartar and caries.

It has been observed that the fact of chewing the equivalent of half a seed for a few minutes while brushing one's teeth helped to better eliminate immediately the tooth film and gave the people the feeling of having a cleaner and sounder mouth than when they brushed their teeth with their dentifrice alone.

That consequently, the invention finds new, beneficial applications helping to better eliminate tooth film, tartar and more generally in order to prevent caries. Such compositions according to the invention improve the cleaning power of chewing gums, tooth pastes and dentifrices.

Consequently, a composition according to the invention can come in the form of chewing gum or else sucking candy.

For preparing a composition according to the invention in the form of chewing gum, people working in the field can advantageously refer to American patents U.S. Pat. No. 3,984,574, U.S. Pat. No. 4,000,320, U.S. Pat. No. 4,151,270 and U.S. Pat. No. 4,415,593.

According to still another method for producing a composition according to the invention, the said composition comes in a form suitable for mouth washes, preferably in liquid form, or else in the form of tooth paste.

A composition according to the invention can come in the form of a mouth wash well known to people working in the field.

A composition according to the invention can come in the form of tooth paste, for example a dentifrice.

For preparing a composition according to the invention in the form of tooth paste or a dentifrice, people working in the field can advantageously refer to American patents U.S. Pat. No. 3,932,606, U.S. Pat. No. 3,935,307, U.S. Pat. No. 3,941,877, U.S. Pat. No. 3,995,024, U.S. Pat. No. 4,181,712, U.S. Pat. No. 5,468,489 and U.S. Pat. No. 5,312,618.

For preparing a composition according to the invention in the form of a composition for fighting tooth film and caries, people working in the field can advantageously refer to American patent U.S. Pat. No. 5,525,330.

A composition according to the invention can come in the form of seeds or extracts or crushed-seed powder, dried or dehydrated or lyophilized, and can be placed in the mouth and chewed at the same time, before or after the person brushes his/her teeth with a dentifrice.

Compositions for Buccal Hygiene for Preventing and Healing Aphthas and their Applications.

A final original and beneficial property of the seeds or crushed seeds has been demonstrated. It was demonstrated during the mouth tests that the seeds and crushed seeds had a beneficial effect on the prevention and healing of aphthas and the acceleration of the healing of sores and aphthas in the mouth; that this property was also accompanied by a quick reduction of the pain related to sores and aphthas in the mouth.

It has been shown that crushed seeds or a seed extract had a beneficial effect concerning the prevention of the appearance of aphthas; that the resorption and cicatrization of the aphthas in the mouth was quicker; that observed at the same time was the rapid disappearance of the pain related to the aphthas, within a half-hour, or just half a day, after eating, chewing, sucking on or masticating the crushed seeds or a protein extract from MORINGA seeds; as opposed to, usually, when no cicatrizing product was used, a few days later.

According to another method of production, a composition according to the invention is characterized by the fact that it comes in a form suitable for the prevention or cicatrization of aphthas and for reducing the pain related to aphthas and other mouth sores, preferably in liquid form.

Such a composition according to the invention helps heal and cicatrize aphthas, aphtoses and mouth sores. It reduces the pain related to these aphthas, aphtoses and mouth sores.

According to a method of producing a composition according to the invention, the said composition comes in a form suitable for mouth washes well known to the people in the field.

A composition according to the invention can be an emulsive, fluid or semi-fluid preparation containing a suspension of the seed powder, and which can, in particular, be used as a product cicatrizing and preventing aphtoses and mouth ailments. This emulsion will be, preferably but not in a limited way, based on various spirits, oils and fatty substances, compatible with the product and with a high percentage of water, able to reach 90%, for, in particular, favoring humidification of the parts of the mouth in contact with the product and facilitating the activity of the preparation. This preparation can be used as a gargle.

Another composition according to the invention can come in the form of sucking candy well known to people in the field.

Another composition according to the invention can come in the form of chewing gum.

For preparing a composition according to the invention in the form of chewing gum, people working in the field can advantageously refer to American patents U.S. Pat. No. 3,984,574, U.S. Pat. No. 4,000,320, U.S. Pat. No. 4,151,270 and U.S. Pat. No. 4,415,593.

A composition according to the invention can come in the form of tooth paste, for example a dentifrice.

For preparing a composition according to the invention in the form of tooth paste or a dentifrice, people working in the field can advantageously refer to American patents U.S. Pat. No. 3,932,606, U.S. Pat. No. 3,935,307, U.S. Pat. No. 3,941,877, U.S. Pat. No. 3,995,024, U.S. Pat. No. 4,181,712, U.S. Pat. No. 5,468,489 and U.S. Pat. No. 5,312,618.

A composition according to the invention can come in the form of crushed seeds or a seed extract or a powder, combined with an effervescent formula such as an effervescent powder or an effervescent tablet obtained by compression, helping to disperse the product in a glass of water.

The seed powder will, in particular, be added to the effervescent mixture by means of various methods of granulation well known to the people in the field, such as that obtained by a dry process, a wet process or by granulation on a fluidized layer of air.

A composition according to the invention can come in the form of crushed seeds, extracts or powders, dried or dehydrated, which can be placed in the mouth and chewed.

A composition according to the invention can come in the form of a non-alcoholic, liquid solution, which can be dabbed on and/or applied to the aphthas or mouth sores.

Compositions for Health Care, in Particular, Health Food

A composition according to the invention can come in the form of health food.

As an illustrative and non-exhaustive example, different ways and process for obtaining a health food will be described below.

It can be either crushed seeds or an extract or a powder produced from seeds which are then mixed in with food or other compositions serving therapeutic purposes or related to hygiene and health.

The health food resulting from the invention can be served as a complement to regular food in a container acting as a dosing dispenser or in a form allowing it to be placed in the mouth.

A composition according to the invention can come in the form of an effervescent formula such as an effervescent powder or an effervescent tablet helping to disperse it.

The seed powder will, in particular, be added to the effervescent mixture by means of various methods of granulation well known to the people in the field, such as that obtained by a dry process, a wet process or by granulation on a fluidized layer of air.

The product comes in the form of an effervescent structure such as an effervescent powder or an effervescent tablet obtained by compression.

A composition according to the invention can come in the form of an emulsion for mouth washes, if these are necessary.

A composition according to the invention can be an emulsive, fluid or semi-fluid preparation containing a suspension of the seed powder, and which can, in particular, be used as a buccal deodorant product. This emulsion will be, preferably but not in a limited way, based on various oils and fatty substances, compatible with the product and with a high percentage of water, able to reach 90%, for, in particular, favoring humidification of the parts of the mouth in contact with the product and facilitating the activity of the preparation. This preparation can be used as a gargle and possibly swallowed.

A composition according to the invention can come in a bio-adhesive form with slow or fast disaggregation, as required, for releasing the quantity necessary for buccal salivation. This composition could also be combined with an effervescent process.

A composition according to the invention can come in the form of crushed seeds or extracts or powders, dried or dehydrated or lyophilized, and can be placed in the mouth and chewed.

A composition helping to eliminate bad breath and allowing fresh breath to last longer, according to the invention, can come in the form of a powder to be mixed with food and be proposed as health food.

As an illustrative and non-exhaustive example, different ways and process for obtaining a health food will be described below.

It can be either crushed seeds or an extract or a powder produced from seeds which are then mixed in with food or other compositions serving therapeutic purposes or related to hygiene and health.

The health food resulting from the invention can be served as a complement to regular food in a container acting as a dosing dispenser or in a form allowing it to be placed in the mouth.

A composition according to the invention can come in the form of an effervescent formula such as an effervescent powder or an effervescent tablet helping to disperse it.

The seed powder will, in particular, be added to the effervescent mixture by means of various methods of granulation well known to the people in the field, such as that obtained by a dry process, a wet process or by granulation on a fluidized layer of air.

The product comes in the form of an effervescent structure such as an effervescent powder or an effervescent tablet obtained by compression.

It can be used as a medicine in the prevention of and the fight against aphthas, aphtoses, mouth sores and ulcers and/or for limiting or eliminating the pain related to these, and/or to help or improve salivation and/or as a health food.

The invention also has as a purpose the use of seeds, crushed seeds or a protein extract from the seeds of a Moringa-type plant for making any one of the compositions covered in this description.

It also relates to any article covered in this description and which contains, in the form of a composition, seeds, crushed seeds or a protein extract from the seeds of a Moringa-type plant.

As non-exhaustive examples of the practical production of compositions or formulations according to the invention, described below are various products or preparations containing at least seeds, crushed seeds or a protein extract from the seeds, with their oil removed or not removed, decorticated or not decorticated, of MORINGA-type plants.

### EXAMPLE 1

A very natural product, in the form of a liquid emulsion-type composition for foot baths, can, for example, present a weighted composition formed with the following aqueous and proteinaceous phases:

| Aqueous phase: Spring water | qsp 1000.00 |
|---|---|
| Dehydrated or fresh crushed Moringa seeds | 20.00 |
| prepared by simply crushing the seeds | |
| without extracting their oil | |
| Preservative and protectant | 0.40 |
| Perfume | 0.30 |

### EXAMPLE 2

A chewing gum for fighting bad breath and preventing caries and aphthas can, for example, have a composition formed mainly with chewing gum, other incorporated substances and a protein extract:

For a tablet weighing 15 grams:

Chewing gum
Anhydrous sodium carbonate
Sorbitol in powdered and solution form (70%)
Mint fragrance or other fragrance
Glucerol (85%)
   Moringa protein extract, with oil not removed: 3 grams.

### EXAMPLE 3

A product in the form of a dentifrice or tooth paste can, for example, have a composition formed with the following excipients and protein extracts:

Excipient:

Sodic carboxymethylcellulose
Vaseline oil
Colloidal silica
Titanium oxide
Sodium laurylsulfate
Demineralized water
Stabilized dicalcic phosphate
Mint fragrance with menthol and mint spirit
   MORINGA seed extract, decorticated and whose oil has not been removed: 20 grams for 100 grams of excipient.

### EXAMPLE 4

Another tooth paste could contain in particular, associated with the Moringaceaes extract or its crushed seeds, as excipients, magnesium carbonate, calcium carbonate, saccharine, essential oils and, if required, Ben oil from the Moringaceaes, vanillin, sodium laurylsulfate, sodic carboxymethylcellulose, glycerol and distilled water or spring water.

### EXAMPLE 5

A product in the form of a divisible effervescent tablet for a solution for gargling and fighting bad breath and whose composition can, for example, be formed with the following conventional excipients:

Excipients on a basis of 10 grams:

Anhydrous citric acid
Anhydrous sodium carbonate
Sodium bicarbonate
Sorbitol powder
Sodic saccharine
Sodium ducosate
Polyvidone
Sodium benzoate
   Crushed seeds, decorticated and oil not removed 1 gram

### EXAMPLE 6

A product in the form of a powder for fighting perspiration and the unpleasant odors related to perspiration can, for example, be a composition formed, in particular, with the following excipients and extracts or crushed seeds:

Excipients:

Talc
Zea Mays
   Decorticated seed extract, dehydrated and oil not removed: 5% of weight of excipients

### EXAMPLE 7

A product in the form of a liquid composition for making a milk-cream and a milk emulsion, with washing qualities, deodorant and soothing, containing, in particular, wheat germ or sweet almond or sesame oil or other oils, glycerin, lanolin, possible hyaluronic derivatives and possibly embryonic ovine extracts and extract or crushed, decorticated Moringa seeds whose oil has not been removed.

### EXAMPLE 8

A product in the form of a soap bar for washing oneself composed of crushed MORINGA seeds, whose oil has not been removed, and lipidic constituents of animal and/or vegetable origin with or without glycerin, with or without an aromatic composition and perfumes, with or without essential oil.

### EXAMPLE 9

A product in the form of a soap bar for washing oneself composed of crushed MORINGA seeds, whose oil has not been removed, lipidic constituents of animal origin, glycerin, possibly lanolin and crushed seeds or a crushed MORINGA seed extract, whose oil has been removed or not removed.

### EXAMPLE 10

A product in the form of a stick or spray, a natural deodorant without alcohol, without aluminum salt, non-allergenic, containing a sudomoderator complex and crushed seeds or a seed extract with a deodorant, soothing and softening effect on the skin.

### EXAMPLE 11

A product in the form of a sucking candy composed of a crushed MORINGA seed extract, whose oil has not been removed, mixed with quick-melting sugar for fighting hyposialia or xerostomia.

### EXAMPLE 12

A product in the form of a protein extract or dehydrated crushed MORINGA seeds which can be proposed as health food in the fight against hyposialia or xerostomia.

### EXAMPLE 13

An effervescent formula that would contain an effervescent mixture of citric acid and sodium bicarbonate, the product, a surface-active agent such as lauryl sulfate and possibly as an option, a perfume able to provide a particular fragrance.

The product would come in the form of an effervescent cube obtained by compression.

### EXAMPLE 14

### Composition of a Body Deodorant Based on Moringa Seeds and Comparative Tests

In accordance with this example, a body deodorant composition based on Moringa seeds was produced. Its deodorant properties were tested on a panel of voluntary individuals and compared with the deodorant properties of an initial composition made the same way but not containing Moringa seeds and a second composition made the same way but in which the Moringa seeds are replaced by a conventional deodorant agent, Triclosan.

A. Materials and Methods

A.1. Compositions

1) Basic Composition not Containing Moringa Seeds

1. Formula Chart

The products tested are four alcoholic deodorants in the form of spray cans.

A conventional deodorant which has no active agent was formulated. This “blank” deodorant served as a standard formula. Into this were incorporated the Moringa seeds and an anti-bacteria agent.

TABLE 1
Perfumed basic deodorant composition (“Blank” deodorant):

| Composition | INCI Name | % W/W |
|---|---|---|
| Ethanol | Alcohol | 87.5 |
| Abil B 8839 | Cyclomethicone | 5 |
| | pentamer | |
| Propylene glycol | Propylene glycol | 2.5 |
| Madison | Perfume | 5 |

2. Packaging

All the deodorants are packaged in aluminum cans with dimensions: dia.=50 mm H=155 mm.

The cans are filled in accordance with the following proportions: 40 g of concentrate and 87 g of butane.

2) Composition Based on Moringa Seeds

The composition of the formulation is shown in Table 2 below.

TABLE 2
Deodorant composition based on Moringa seeds

| Composition | INCI Name | % W/W |
|---|---|---|
| Ethanol | Ethanol | 83.5 |
| Abil B 8839 | Cyclomethicone | 5 |
| | pentamer | |
| Propylene | Propylene glycol | 2.5 |
| glycol | | |
| Moringa seeds | Moringa Oleifera | 4 |
| Perfume | Madison | 5 |

Procedure

1—Mix the ethanol with the perfume using magnetic agitation.

2. Crush the seeds with a pestle. Mix the powder obtained with the propylene glycol.

3. Mix this paste with the alcoholic solution. Then filter with a 150 μaluminum filter.

4. Add the Abil® B 8839 to the mixture and agitate for ten minutes.

3) Composition Based on Triclosan

The formulation of the Triclosan-based composition is shown in Table 3 below.

TABLE 3
Deodorant composition containing Triclosan

| Composition | INCI Name | % W/W |
|---|---|---|
| Ethanol | Ethanol | 87.25 |
| Abil B 8839 | Cyclomethicone | 5 |
| | pentamer | |
| Propylene | Propylene glycol | 2.5 |
| glycol | | |
| Irgasan ™ 300 | Triclosan | 0.25 |
| DP | | |
| Perfume | Madison | 5 |

Procedure

1—Mix the ethanol with the perfume and the Irgasan™ 300 DP using magnetic agitation.

2—Add the Abil B 8839 and the propylene glycol to the preceding phase.

3—Agitate for ten minutes.

A.2. Protocol for Comparative Test

Setting Up the Test

1. The Panel

It is composed of eight volunteers from the company three of which are of the masculine sex and five of the feminine sex. The average age is equal to 38.8 years.

2. The Place

The persons participating in this survey applied the products distributed to them in their homes under normal conditions of use.

3. Duration

The tests were conducted over a period of four days for each of the tests.

Methodology for the Test

The three deodorant or deodorizing substances whose respective composition is shown in Tables 1 to 3 above were produced.

After this formulation step, three comparison tests were carried out in the following order:

1—“Blank” deodorant versus Irgasan™-based deodorant. This test is used to validate the test.
2—Moringa-based deodorant with respect to the “blank” deodorant. This test is used to evaluate the anti-odor effectiveness of the Moringa seeds.
3—Moringa-based deodorant versus the Irgasan™-based deodorant. This test is used to compare the effectiveness of the seeds with that of the Irgasan™.
   These test were conducted “blind”, i.e. the panelists did not know the nature of the deodorants. To do this, each deodorant is coded differently. Each subject is his/her own witness.
“blank” deodorant: 02CB217
Irgasan™-based deodorant: 02CB053
Moringa-based deodorant: 02CB656
   Protocol
   Objective: Each individual on the panel was given two body deodorants that they tested in order to determine their effectiveness in fighting unpleasant body odors. This study will show the possible differences between the two products, in accordance with the following protocol:
1) D1: In the morning, on clean, dry skin, spray one of the deodorants being tested onto one of the arm pits, and the other deodorant onto the other arm pit.
2) D1+8 hours: In the evening, smell the arm pits one after the other and note the perception for both arm pits in the table below (from 0 to 4).
3) Repeat this test three days in a row, always applying the same deodorant to the same arm pit.
   Notation Scale for Marks Given:

0=no effectiveness—only unpleasant body odor is perceived

1=mediocre effectiveness

2=average effectiveness

3=good effectiveness

4=very good effectiveness—no bad odor is perceived

B. Results

With the results, the average for the marks given by each individual on the panel and for each of the tests is calculated. These results were used to plot histograms representing the deodorant effectiveness perceived for each panelist.

Each comparison was treated statistically in order to determine the statistical value of the results.

The test used is the Wilcoxon test for matched series. It is a non-parametric test. This test is used classically in sensory analysis to compare two different products evaluated by the same jury.

1. Comparative Study for “Blank” Deo/Moringa-Based Deo

This test served to evaluate the effectiveness of the Moringa seeds. The results are shown in Table 4 below, in the form of the average of the marks given and the standard deviation with respect to the average.

TABLE 4

| | |
|---|---|
| “Blank” Deodorant | Moringa-based Deodorant |

| Panelists | Average | Average |
|---|---|---|
| 1 | 1.5 | 3 |
| 2 | 1.5 | 2.25 |
| 3 | 3.75 | 4 |
| 4 | 2.5 | 4 |
| 5 | 3.25 | 3.75 |
| 6 | 1.75 | 2.5 |
| 7 | 1 | 1.75 |
| 8 | 0.5 | 1.75 |

According to the results in Table 4 above, it can be seen that the Moringa-based deodorant has good effectiveness, the overall average being 2.875. Concerning the “blank” deodorant, the effectiveness perceived is mediocre (overall average=1.75).
The statistical test carried out on this comparison shows that the difference between the two samples is significant.
Statistical Results of the Comparison Between Moringa-Based Deo/“blank” Deo
Signed Wilcoxon Test/Bilateral Test:
Remark: The variance in the Wilcoxon Test was calculated taking into account the absolute differences ex oequo.
In the case of small evaluation groups (<25), reference can be made to a table published in Siegel (S.), 1956: Nonparametric Statistics for the Behavioural Sciences. McGraw-Hill Kogakusha, Tokyo, 312 p. The table of critical values for the Wilcoxon Test is given on p. 254.
Value observed in Wilcoxon Test: 0.000
Expectancy in Wilcoxon Test: 18.000
Variance in Wilcoxon Test: 50.375
The Wilcoxon Test is reduced centered and tested with respect to the normal law.
Value observed in reduced centered statistics z: −2.536
Associated P-value: 0.006
As the test is bilateral, the p-value is compared to the threshold of significance: alpha/2=0.025
Critical value in reduced centered statistics z: 1.960
Conclusion
At the total significance threshold alpha=0.050, the assumption of zero for no difference between samples can be rejected.
In conclusion, the difference between samples is significant.
2. Results of the Comparison Between Irgasan™-Based Deo/“Blank” Deo

The results are shown in Table 4 below, in the form of the average of the marks given and the standard deviation with respect to the average.

TABLE 5

| | |
|---|---|
| “Blank” Deo | Irgasan ™-based Deo |

| Panelists | Average | Average |
|---|---|---|
| 1 | 1.67 | 3.33 |
| 2 | 2.33 | 3.00 |
| 3 | 1.67 | 3.00 |
| 4 | 2.00 | 2.33 |
| 5 | 1.00 | 1.67 |
| 6 | 1.00 | 2.67 |
| 7 | 2.33 | 3.00 |
| 8 | 1.67 | 3.00 |

According to the results in Table 5 above, it can be observed that the Irgasan™-based deodorant has an effectiveness greater than that of the “blank” deodorant. The effectiveness of the Irgasan™-based deodorant is good but that of the “blank” deodorant is mediocre.

The panelists are valid. The other tests can continue with the same panel and the same protocol.

Statistical Results of the Comparison Between “Blank” Deo/Irgasan-Based Deo.

Signed Wilcoxon Test/Bilateral Test:

Remark: The variance in the Wilcoxon Test was calculated taking into account the absolute differences ex oequo.

In the case of small evaluation groups (<25), reference can be made to a table published in Siegel (S.), 1956: Nonparametric Statistics for the Behavioural Sciences. McGraw-Hill Kogakusha, Tokyo, 312 p. The table of critical values for the Wilcoxon Test is given on p. 254.

Value observed in Wilcoxon Test: 1.000

Expectancy in Wilcoxon Test: 18.000

Variance in Wilcoxon Test: 50.750

The Wilcoxon Test is reduced centered and tested with respect to the normal law.

Value observed in reduced centered statistics z: −2.386

Associated P-value: 0.009

As the test is bilateral, the p-value is compared to the threshold of significance: alpha/2=0.025

Critical value in reduced centered statistics z: 1.960

Conclusion

At the total significance threshold alpha=0.050, the assumption of zero for no difference between samples can be rejected.

In conclusion, the difference between samples is significant.

Statistical Results

We assume, in the assumption of zero H₀, that no difference is significant between the samples with the risk α=5%.

The H₀ assumptions is rejected if p≦0.05. Here, as the test is bilateral, the p-value is compared to the significance threshold: α/2=0.025.

The statistical test shows that the difference between the samples is significant.

This statistical treatment confirms thoroughly that the panelists perceived a difference between the two samples and that the panel is therefore valid.

3. Results of the Comparison Between Irgasan™-Based Deo/Moringa-Based Deo

This test confirmed the preceding test and showed that the Moringa has an effectiveness comparable with that of Irgasan™. The results are shown in Table 6 below, in the form of the average of the marks given and the standard deviation with respect to the average.

TABLE 6

| | |
|---|---|
| Moringa-based Deo | Irgasan ™-based Deo |

| Panelists | Average | Average |
|---|---|---|
| 1 | 2 | 3 |
| 2 | 2.66 | 3 |
| 3 | 3.66 | 3 |
| 4 | 2.66 | 3 |
| 5 | 2.66 | 3 |
| 6 | 3.66 | 3.33 |
| 7 | 3.33 | 3.33 |
| 8 | 1 | 2.66 |

It can be seen from the results shown in Table 6 above that the Irgasan™-based deodorant was noted in almost the same way by all the panelists. They judged that the effectiveness of the deodorant was good. Concerning the Moringa-based deodorant, the deodorant effectiveness perceived varies with the panelists. However, it remains relatively good. The overall average is 2.70. As the averages are comparable, it is deduced that the difference between these two deodorants is not significant.
It can be concluded from this that the Moringa seeds have an effectiveness comparable with those of Irgasan™.
To confirm this assumption, the Wilcoxon Test was conducted. The statistical test shows that the difference between the samples is not significant. In other words, it can be said that the jury did not perceive any real differences, consequently, that the Moringa would have an acceptable effectiveness against bad odors.
Statistical Results of the Comparison Between Irgasan™-Based Deo/Moringa-Based Deo
Signed Wilcoxon Test/Bilateral Test:
Remark: The expectancy and the variance in the Wilcoxon Test were calculated taking into account the zero differences.
Remark: The variance in the Wilcoxon Test was calculated taking into account the absolute differences ex oequo.
In the case of small evaluation groups (<25), reference can be made to a table published in Siegel (S.), 1956: Nonparametric Statistics for the Behavioural Sciences. McGraw-Hill Kogakusha, Tokyo, 312 p. The table of critical values for the Wilcoxon Test is given on p. 254.
Value observed in Wilcoxon Test: 8.000
Expectancy in Wilcoxon Test: 17.500
Variance in Wilcoxon Test: 50.250
The Wilcoxon Test is reduced centered and tested with respect to the normal law.
Value observed in reduced centered statistics z: −1.340
Associated P-value: 0.090
As the test is bilateral, the p-value is compared to the threshold of significance: alpha/2=0.025
Critical value in reduced centered statistics z: 1.960
Conclusion
At the total significance threshold alpha=0.050, the assumption of zero for no difference between samples cannot be rejected.
In conclusion, the difference between samples is not significant.
C. Conclusions Concerning the Results of Example 14

In a general way, positive points with regard to the effectiveness of the Moringa seeds appeared. Among the tests showing whether or not the seeds had an action against unpleasant body odors, the results were convincing and prove that the seeds are effective against offensive odors.

### EXAMPLE 15

### Test 1 Concerning the Properties of Moringa Seeds with Respect to Anti-Foaming Power and Salivation

A. Materials and Methods

A.1: Preparation of Tooth Pastes

| Trade Name | INCI Name | % weight |
|---|---|---|
| Glycerine | Glycerine | 6 |
| Blanose 7MXF | Cellulose gum | 0.4 |
| Sorbitol | Sorbitol | 4.5 |
| Moringa | Moringa oleifera | 3 |
| Water | Aqua | 5.72 |
| Sorbitol | Sorbitol | 6 |
| Sodium Saccharinate | Sodium Saccharin | 0.28 |
| Sodium Benzoate | Sodium Benzoate | 0.3 |
| Sodium | Sodium | 0.76 |
| Monofluorophosphate | Monofluorophosphate | |
| Sorbitol | Sorbitol | 6 |
| Water | Aqua | 3 |
| Syloblanc 81 | Silica | 4 |
| Syloblanc 34 | Silica | 12.5 |
| Titanoxyd P25 | Titanium Dioxide | 1.5 |
| Sipon LC98 | Sodium Lauryl Sulfate | 1.5 |
| Menthol | Menthol | 1 |
| Irgasan DP 300 | Triclosan | 0.25 |
| Water | Aqua | 4 |

Procedure:
1—Mix the blanose with the glycerine until uniformity is reached.
2—Pour in phase B, stirring moderately for 15 minutes.
3—Crush the Moringa seeds with the sorbitol from phase C. Add the paste obtained to the water and stir for five minutes.
4—Filter with a 150 μaluminum filter.
Pour this solution into the above mixture and stir vigorously for 30 minutes.
5—Add phase D previously heated to 40° C. Stir vigorously for 15 minutes
6—Pour the gel obtained into a mortar.
Incorporate phase E little by little and pound in order to obtain a white cream.
7'Prepare phase F as follows:
dissolve the Irgasan in the perfume
dissolve the sipon in the water
   Mix together the two phases then pour the result obtained into the paste.
   The tooth paste is packaged in 100 ml plastic tubes 45 mm in diameter and 120 mm in length, provided with an outlet opening 5 mm in diameter. Each tube was filled with 60 g of white tooth paste.
   A.2: Execution of Test
   Number of Subjects
   The study was conducted with eight volunteers four of which were of the masculine sex and four of the feminine sex. The average age of this panel was 29.6 years of age.
   Place
   Once the products were distributed, the volunteers carried out the test at home in accordance with normal conditions of use.
   Duration
   The volunteers used the respective samples for eight days, applied twice daily (morning and evening).
   Reference
   The test was conducted “blind”. The products were supplied in coded form so as not to differentiate the two dentifrices.
“Pacebo” dentifrice: 02CB600
Dentifrice with Moringa: 02CB073
   A3: Methodology
   A clearly-defined protocol was established to carry out these tests under standard conditions. At the end of each test, two subjective evaluation questionnaires concerning the tolerance to the product and its effectiveness are given to the volunteers (these questionnaires are distributed to the subjects at the end of the test so as not to influence their judgment). These are lay and spontaneous subjects
   The first questionnaire is based on the rheological behavior of the tooth pastes. The second one refers to the properties of the Moringa seeds and in particular to the following claims:
Modification of the state of salivation
Bitterness of the product
   B. Results
   Viscosity
   First of all, the individuals tested (panelists) agreed that the standard tooth paste and the Moringa-based tooth paste had similar viscosity, the Moringa-based tooth paste being judged slightly more fluid.
   The results show that, out of the eight panelists, 4 judged the Moringa-based tooth paste as fluid, against 3 for the standard tooth paste.
   Out of the eight panelists, 4 panelists judged the Moringa-based tooth paste as slightly viscous, and also 4 panelists judged the standard tooth paste as slightly viscous.
   Foaming Power
   The foaming power of, respectively, the standard tooth paste and the Moringa-based tooth paste was tested with eight panelists.
   The results show that the individuals tested estimated that the Moringa-based tooth paste had low foaming power, which shows that the Moringa seeds, the crushed seeds or a protein extract from Moringa seeds is able to reduce or break the foaming effect of the standard tooth paste's composition.
   Out of the eight panelists, 7 judged the Moringa-based tooth paste as having low foaming power and 1 determined that it had average foaming power.
   On the other hand, for the reference tooth paste, without Moringa seeds, the tooth paste's foaming power was judged as being average for 4 panelists and high for the other 4 panelists.
   The volume of foam produced when brushing is in this case small for the Moringa-based tooth paste. On the other hand, the standard tooth paste has high foaming power.
   Bitterness
   One of the organoleptic properties of the Moringa seeds is their bitter taste. But according to the results obtained after judgment by the panelists, this bitterness is not perceived when the seeds are incorporated into a buccal composition.
   Salivation
   Another very important characteristic of the Moringa seeds was demonstrated during the test: modification of the state of the saliva when brushing the teeth.
   Out of the eight volunteers tested (panelists), 4 judged that the Moringa-based tooth paste had caused, during brushing, greater salivation than when brushing with the reference tooth paste, without Moringa seeds.

### EXAMPLE 16

### Test 2 Concerning the Effectiveness of the Moringa Seeds with Respect to Breath and Salivation

A. Materials and Methods

Panel of Volunteers

The test was carried out on 20 volunteers, 16 of which were of the feminine sex and 4 of the masculine sex.

Execution of Study

On D0:

The volunteers come to the laboratory in the morning, having brushed their teeth two days earlier.

The information sheet reviews for them the successive steps of the test

They sign the two forms: consentment sheet and information sheet.

Clinical examination, by the dentist in charge of the study, who gives a mark for mouth odor.

Measurement of the time required for a lump of surgar to melt in the mouth.

Ten volunteers brush their teeth with product M, the other ten with product N.

Another clinical examination, by the dentist in charge of the study, who evaluates the same parameters as previously.

Another measurement of the time required for a lump of surgar to melt in the mouth.

The volunteers fill in a subjective evaluation questionnaire concerning the immediate effect.

The volunteers are instructed not to use other buccal hygiene products, not to remove any tartar and to maintain their eating habits (in particular tea and coffee) and consumption of tobacco. Except on the days of the study when they must come without having drunk any coffee or smoked.

On D7:

Clinical examination, by the dentist in charge of the study, who evaluates the same parameters as on D0.

Measurement of the time required for a lump of surgar to melt in the mouth.

The volunteers fill in a subjective evaluation questionnaire and sign their study-termination sheet.

Freshness of Breath

The dentist evaluates the mouth odor and the amount of tooth film of the volunteers in accordance with the following scales:

Intensity of mouth odor—

1: imperceptible
2: . . .
. . . / . . .
8: . . .
9: extremely strong
   Sugar Melting Time
   After brushing with, respectively, the reference tooth paste containing no Moringa seeds and the Moringa seed based tooth paste, whose composition is described in example 15, the time require for sugar to melt in the mouth was measured.
   The sugar melting time serves to evaluate the salivation of the individuals being tested. The greater the salivation, the shorter the melting time.
   The sugar melting time is used to quantify accurately la salivation of the individuals undergoing the test.
   B. Results

B.1. Effect of the Moringa Seeds on Breath

TABLE 7
Results of the effect of the Moringa seeds on breath
At the left: Reference tooth paste
At the right: Moringa-based tooth paste

| Standard tooth paste | | | | | | Moringa-based tooth paste | | | | | |
|---|---|---|---|---|---|---|---|---|---|---|---|
| | intensity | | | | | | intensity | | | | |
| | of mouth odor | | | | | | of mouth odor | | | | |
| | | after 1st | after 6 | Δ after 1st | Δ after 6 | | | after 1st | after 6 | Δ after 1st | Δ after 6 |
| | ini- | appli- | days of | appli- | days of | | ini- | appli- | days of | appli- | days of |
| volunteer | tial | cation | treatment | cation | treatment | volunteer | tial | cation | treatment | cation | treatment |
| 1 | 2 | 1 | 1 | −1 | −1 | 2 | 2 | 1 | 1 | −1 | −1 |
| 3 | 1 | 1 | 1 | 0 | 0 | 4 | 3 | 2 | 1 | −1 | −2 |
| 5 | 2 | 1 | 1 | −1 | −1 | 6 | 2 | 2 | 3 | 0 | 1 |
| 7 | 2 | 1 | 1 | −1 | −1 | 8 | (1)* | (1)* | (1)* | VM | VM |
| 9 | 3 | 1 | 2 | −2 | −1 | 10 | 3 | 2 | 1 | −1 | −2 |
| 11 | 2 | 2 | 1 | 0 | −1 | 12 | 1 | 1 | 1 | 0 | 0 |
| 13 | 4 | 2 | 3 | −2 | −1 | 14 | 3 | 2 | 1 | −1 | −2 |
| 15 | 2 | 2 | 1 | 0 | −1 | 16 | 3 | 2 | 2 | −1 | −1 |
| 17 | 1 | 1 | 1 | 0 | 0 | 18 | 1 | 1 | 1 | 0 | 0 |
| 19 | 1 | 1 | 1 | 0 | 0 | 20 | 2 | 1 | 1 | −1 | −1 |
| Average | 2.0 | 1.3 | 1.3 | −0.7 | −0.7 | Average | 2.2 | 1.6 | 1.3 | −0.7 | −0.9 |
| Mean | 2.0 | 1.0 | 1.0 | −0.5 | −1.0 | Mean | 2.0 | 2.0 | 1.0 | −1.0 | −1.0 |
| Minimum | 1.0 | 1.0 | 1.0 | −2.0 | −1.0 | Minimum | 1.0 | 1.0 | 1.0 | −1.0 | −2.0 |
| Maximum | 4.0 | 2.0 | 3.0 | 0.0 | 0.0 | Maximum | 3.0 | 2.0 | 3.0 | 0.0 | 1.0 |
| SEM | 0.3 | 0.2 | 0.2 | 0.3 | 0.2 | SEM | 0.3 | 0.2 | 0.2 | 0.2 | 0.4 |
| IC 95% | 0.7 | 0.3 | 0.5 | 0.6 | 0.3 | IC 95% | 0.6 | 0.4 | 0.5 | 0.4 | 0.8 |

The results are shown in table 7 above.
Table 7 shows the individual results for the intensity of mouth odor as well as the related descriptive statistics (average, mean, minimum, maximum, standard deviation with respect to average, and interval of confidence at 95%).
The results show that the Moringa seeds have a beneficial effect on breath by reducing the intensity of mouth odor.
In Table 7, the average of the panelists' notations is in this case, respectively, −0.7 for the standard tooth paste used as a reference and −0.9 for the Moringa-based tooth paste.
B.2. Effect of the Moringa Seeds on Salivation

The results are shown in Table 8 below. To the left are shown the results obtained with the standard tooth paste without Moringa seeds. To the right are shown the results obtained with the Moringa seed based tooth paste.

| | |
|---|---|
| Standard | Moringa |

| | sugar melting time | | | | | | sugar melting time | | | | |
|---|---|---|---|---|---|---|---|---|---|---|---|
| | in seconds | | | | | | in seconds | | | | |
| | | after 1st | after 6 | Δ after 1st | Δ after 6 | | | after 1st | after 6 | Δ after 1st | Δ after 6 |
| | ini- | appli- | days of | appli- | days of | | ini- | appli- | days of | appli- | days of |
| volunteer | tial | cation | treatment | cation | treatment | volunteer | tial | cation | treatment | cation | treatment |
| 1 | 10 | 15 | 15 | 1 | 1 | 2 | 38 | 18 | 20 | 12 | [symbol] |
| 3 | 10 | 24 | 17 | 44 | 7 | 4 | 54 | 24 | [symbol] | 63 | [symbol] |
| 5 | 20 | 20 | 27 | 2 | 1 | 6 | 10 | 23 | 16 | 17 | 24 |
| 7 | 40 | 50 | 48 | 8 | 0 | 8 | (10) [symbol] | (10) [symbol] | (10) [symbol] | VM | VM |
| 9 | 47 | 24 | 0 | 7 | 8 | 10 | 14 | 27 | 27 | 13 | 13 |
| 11 | 17 | 28 | 24 | 14 | 4 | 12 | 82 | [symbol] | 24 | 2 | 8 |
| 13 | 10 | 14 | 15 | 4 | 5 | 14 | 20 | 16 | 10 | 4 | 10 |
| 15 | 10 | 14 | 13 | 4 | 5 | 16 | 20 | 20 | 16 | [symbol] | [symbol] |
| 17 | 20 | 20 | 17 | 0 | 3 | 18 | 45 | 34 | 10 | [symbol] | 20 |
| 19 | 94 | 60 | [symbol] | 22 | 60 | 20 | 15 | 17 | 16 | 2 | 1 |
| Average | 27.5 | 28.4 | 21.3 | 0.0 | 6.2 | Average | 30.7 | 22.7 | 18.4 | 8.0 | 9.4 |
| Mean | 17.5 | 24.0 | 17.0 | 1.0 | 1.0 | Mean | 28.0 | 21.0 | 17.5 | [symbol] | 0.0 |
| Minimum | 10.0 | 14.0 | 0.0 | 22.0 | 60.0 | Minimum | 14.0 | 10.0 | 10.0 | 33.0 | 26.0 |
| Maximum | 94.0 | 60.0 | 48.0 | 44.0 | 7.0 | Maximum | 54.0 | 34.0 | 27.0 | 13.0 | 13.0 |
| SEM | 7.0 | 6.6 | 3.8 | 3.1 | 6.2 | SEM | 4.5 | 2.1 | 1.0 | 4.3 | 4.5 |
| IC 95% | 17.8 | 12.7 | 8.2 | 7.1 | 13.9 | IC 95% | 10.5 | 5.0 | 4.5 | 9.9 | 10.6 |
| | p = | 0.78035 | 0.33983 | | | | p = | 0.09908 | 0.07385 | | |

Table 8 above shows the individual results for the sugar melting time as well as the related descriptive statistics (average, mean, minimum, maximum, standard deviation with respect to average, and interval of confidence at 95%).
After the standard tooth paste has been used, the sugar's average melting time does not vary. It is 27.5 seconds before use and 28.4 seconds after being used for the first time. Differences are observable only after 6 days of using the standard tooth paste.
On the other hand, the sugar's average melting time decreases significantly as soon as the Moringa seed based tooth paste is used for the first time. The average melting time is 30.7 seconds before use and 18.4 seconds after being used for the first time.
After 6 days, the tooth paste based on Moringa seeds even allows a reduction in the average sugar melting time down to 18.4 seconds.
The results of the test show that the Moringa seed based tooth paste strongly stimulates salivation, which is considerably helpful to buccal hygiene.

1. A composition used as a deodorant or for buccal or body hygiene, or for health care, characterized by the fact that it contained seeds or crushed seeds or a protein extract from the seeds of a Moringa-type plant.

2. A composition as described in claim 1, characterized by the fact that the seeds are decorticated or not decorticated.

3. A composition as described in one of the claims 1 and 2, characterized by the fact that it contains seeds, crushed seeds or proteinaceous seed extract whose oil as been removed or not removed.

4. A composition as described in one of the claims 1 to 3, characterized by the fact that it is a deodorant and/or deodorizing composition.

5. A composition as described in claim 4, characterized by the fact that the deodorant and/or deodorizing composition has an additional hydrating, soothing, fatigue-relieving action and does not foam.

6. A composition as described in claim 4, characterized by the fact that it is a topic composition in the form of a liquid cleaning solution used as such or in the form of a cloth, a wad of cotton or a cellulose cleaning or a fabric wetted with the deodorant and/or deodorizing solution; a milk fluid, a cream, a soap bar, a liquid or solid soap, a skin-care bar, a lotion or a foam.

7. A composition as described in claim 4, characterized by the fact that it is a powder, placed, if need be, in a container fitted with a dispersion mechanism or used as is; or used as a mixture for baths, for salts, talcs; or used in the composition of special products for preparing feet for physical activity or for sports; or used in the composition of dry shampoos.

8. A composition as described in claim 4, characterized by the fact that it is a stick, a gel or a bi-gel.

9. A composition as described in claim 4, characterized by the fact that it is a fluid or semi-fluid emulsion or semi-emulsion.

10. A composition as described in claim 4, characterized by the fact that it comes in the form of a product entering into the composition of anti-odor fabrics, clothing, products and materials.

11. A composition as described in claim 4, characterized by the fact that it can be incorporated into the composition of absorbent and anti-odor products.

12. A composition as described in one of the claims 1 to 3, characterized by the fact that it is a composition favoring and/or improving salivation.

13. A composition as described in one of the claims 1 to 3, characterized by the fact that it is a composition eliminating bad breath and allowing a fresh breath to last longer.

14. A composition as described in one of the claims 1 to 3, characterized by the fact that it is a composition helping to eliminate tooth film, tartar, preventing the occurrence of caries and improving the cleaning power of chewing gums, tooth pastes and dentifrices.

15. A composition as described in one of the claims 1 to 3, characterized by the fact that it is a composition preventing the appearance of aphthas, aphtoses and mouth sores; helping to heal and cicatrize aphthas, aphtoses and mouth sores; and reducing the pain related to these aphthas, aphtoses and mouth sores.

16. A composition as described in claim 15, characterized by the fact that it can be a non-alcoholic liquid solution which can be dabbed on and/or applied to the aphthas, aphtoses and mouth sores.

17. A composition as described in claims 12 to 15, characterized by the fact that it is a chewing gum, a sucking candy, a tooth paste or a dentifrice.

18. A composition as described in claims 12 to 15, characterized by the fact that it is in effervescent form such as an effervescent powder or an effervescent tablet.

19. A composition as described in claims 12 to 15, characterized by the fact that it comes in dry or dehydrated or lyophilized form and can be placed in the mouth and/or swallowed.

20. A composition as described in claims 1 to 3, characterized by the fact that it is a product that can come in the form of a health food.

21. The use of seeds or crushed seeds or a protein extract from the seeds of a Moringa-type plant for making a composition as described in any one of the claims 1 to 20.
